(19) United States
(12) Patent Application Publication
(10) Pub. No.: US 2018/0299403 A1
(43) Pub. Date: Oct. 18, 2018

(54) DEVICES, SYSTEMS, AND METHODS FOR THE DETECTION OF ANALYTES

(71) Applicant: PROTEOSENSE, Columbus, OH (US)

(72) Inventors: Mark T. Byrne, Loveland, OH (US); Erandi K. De Silva, Columbus, OH (US); Jeremiah Schley, Columbus, OH (US)

(21) Appl. No.: 15/524,723

(22) PCT Filed: Nov. 9, 2015
(86) PCT No.: PCT/US2015/059710

§ 371 Date: May 5, 2017

Related U.S. Application Data
(60) Provisional application No. 62/077,056, filed on Nov. 7, 2014.

(51) Int. Cl. G01N 27/414; G01N 33/569; H01L 29/20; H01L 29/205

(52) U.S. Cl. CPC G01N 27/4145; G01N 33/56911; G01N 2333/195; H01L 29/205; H01L 29/2003

(57) ABSTRACT

Disclosed are devices, systems, and methods for the rapid and accurate detection of analytes, including Salmonella.

### TECHNICAL FIELD

This application relates generally to devices, systems, and methods for the quantification of analytes.

### BACKGROUND

The rapid detection of analytes is important in a variety of contexts. For example, food-borne pathogens, such as Salmonella bacteria, represent a significant public health concern. It is estimated that Salmonella enterica is responsible for approximately 1 million infections/year in the U.S. alone. Salmonella is primarily transmitted through contaminated food sources. The accurate detection of Salmonella during harvest, food processing, manufacturing and shipping are critical to preventing the spread of Salmonella. However, existing diagnostics (PCR, ELISA) are time consuming (>48 hrs to result) and require long incubation times (>12 hrs).

Improved rapid devices, systems, and methods for the detection and quantification of analytes, such as Salmonella, offer the possibility to reduce the spread of food-borne illnesses.

### SUMMARY

Provided herein are devices, systems, and methods for the detection and quantification of analytes. The devices, systems, and methods described herein can be used to accurately and rapidly detect and quantify analytes of interest in samples.

Devices and systems for detecting analytes can include a sensor cartridge and a cartridge reader. The sensor cartridge can comprise a chip that includes an active sensor (e.g., one or more FET-based sensors configured to detect one or more analytes of interest), and optionally a reference sensor (e.g., one or more reference sensors configured to provide for signal-noise improvement). In certain embodiments, the sensor cartridge can include two or more active sensors (e.g., at least 3, at least 5, at least 10, at least 25, at least 50, or at least 100 active sensors) disposed on the chip. When more than one active sensor is present in the chip, all the sensors can be configured to detect the same analyte of interest or different analytes of interest. Active sensors may be configured in a variety of suitable configurations. For example, active sensors can be configured individually, in a two dimensional array, in a three dimensional array, or in a Whetstone bridge configuration.

In some embodiments, the sensor cartridge can comprise a sample handling apparatus configured to accept samples of various volumes (e.g., a sample of one analytical unit as specified in the FDA Biological Assessment Manual, or 25 grams). The sample handling apparatus may be configured to induce the sample material to move across one or more sensors in the cartridge (e.g., a flow cell) to allow the entirety of the sample volume to contact the one or more sensors in the cartridge.

The active sensor(s) in the device can comprise a substrate (e.g., sapphire); a channel disposed on the substrate, wherein the channel is substantially impermeable to ions under physiological conditions; a source electrode and a drain electrode electrically connected to the channel, wherein the source electrode and the drain electrode are formed to be separate such that the channel forms a path for current flow between the source electrode and the drain electrode; and a recognition element for the analyte of interest immobilized on the surface of the channel. The distance between the recognition element and the channel can be configured such that association of the analyte of interest with the recognition element induces a change in the electrical properties of the channel. In some applications, the dimensions of the analyte of interest may be large relative to the area of channel, causing changes to the electrical properties of the FET (e.g., a change in capacitance). The reference sensor(s) in the device can comprise a substrate; a channel disposed on the substrate, wherein the channel is substantially impermeable to ions under physiological conditions; a source electrode and a drain electrode electrically connected to the channel, wherein the source electrode and the drain electrode are formed to be separate such that the channel forms a path for current flow between the source electrode and the drain electrode; and a passivating layer disposed on the surface of the channel.

The cartridge reader can be any suitable device configured to receive the sensor cartridge, and interrogate the sensors (e.g., the one or more active sensors and one or more reference sensors, when present) to detect an analyte in close proximity to the active sensors (e.g., within 50 nm of the active sensors, within 25 nm of the active sensors, within 10 nm of the active sensors, within a Debye length of the active sensors, in contact with the active sensors, and/or bound to or otherwise associated with the recognition element of the active sensors). The cartridge reader can include a receiving unit configured to physically receive the sensor cartridge, the receiving unit further comprising receiving components that operably (e.g. electrically) connect to the active sensor and the reference sensor; and a microprocessor configured to analyze an electrical property of one or more active sensors and, in some cases, an electrical property of the reference sensor to detect the analyte of interest.

The cartridge reader can further comprise a display configured to display an output from the microprocessor related to the detection of the analyte of interest. The cartridge reader can further comprise a communication interface configured to transmit data from the microprocessor to a remote computing device wirelessly (e.g., a GSM cellphone connection, a Wi-Fi module, Bluetooth module, or packet radio) or by direct wired connection (e.g. a USB port, or an Ethernet port)

In some embodiments, the cartridge reader can further comprise an input device configured to provide user inputs to the microprocessor. For example, the input device can comprise a barcode scanner, one or more input keys, and/or a touchscreen. In these embodiments, the microprocessor can be configured to correlate user inputs from the input device to an electrical property of the active sensor, an electrical property of the reference sensor, an output from the microprocessor related to the detection of the analyte of interest, or a combination thereof. In this way sensor readings can be correlated with samples being analyzed and analyte presence/absence and or concentration determined and communicated.

In some embodiments the cartridge reader further comprises a real-time-clock. In these embodiments, the microprocessor can be further configured to correlate sensor readings with the date and time. This way the data transmitted from the cartridge reader to a remote computing device can include the sensor readings and time of the test.

In some embodiments, the cartridge reader can further comprise a global positioning receiver (GPS). In these embodiments, the microprocessor can be further configured to correlate sensor readings with the physical location of the reader (e.g. the coordinates in a field of produce). This way the data transmitted from the cartridge reader to a remote computing device can include sample location in addition to the sensor readings.

In some embodiments the cartridge reader further comprises a digital camera. In these embodiments, the microprocessor can be further configured to correlate sensor readings with an image of the sample or the spot where the sample was collected. This way the data transmitted from the cartridge reader to a remote computing device can include the sensor readings and an image.

### DESCRIPTION OF DRAWINGS

FIG. 1 is a perspective view of elements of a system for the detection of analytes.

FIG. 2 is a cross-sectional side view of an active sensor that can be disposed on a chip present in the sensor cartridge.

FIG. 3 is a cross-sectional side view of an active sensor which includes a channel that comprises a Group III-nitride heterojunction.

FIG. 4 is a block diagram illustrating elements of the system described herein.

FIG. 5 is a schematic illustration of methods of detecting analytes using the systems described herein.

FIG. 6 is an illustration of a sensor cartridge.

FIG. 7A, FIG. 7B, and FIG. 7C illustrate potential layouts of active sensors on chips.

FIG. 8 is a schematic illustration of a chip for use in a sensor cartridge.

FIG. 9 is a schematic illustration of the sample in contact with a sensor.

FIG. 10 is a plot illustrating the change in a measured sensor electrical property (current versus threshold voltage) in response to exposure of the sensor towards PBS and a sample contaminated with Salmonella.

FIG. 11 is a plot illustrating the different response in a measured sensor electrical property to a sample contaminated with live Salmonella and dead Salmonella.

FIG. 12A, FIG. 12B, and FIG. 12C are schematic illustrations of example sample handling apparatus that can accept fluid samples of various volumes and induce the sample material to move across one or more sensors in the cartridge (e.g., a flow cell) to allow the entirety of the sample volume to contact the one or more sensors in the cartridge.

### DETAILED DESCRIPTION

Provided herein are devices, systems, and methods for the detection and quantification of analytes. The devices, systems, and methods described herein can be used to accurately and rapidly detect and quantify analytes of interest in samples.

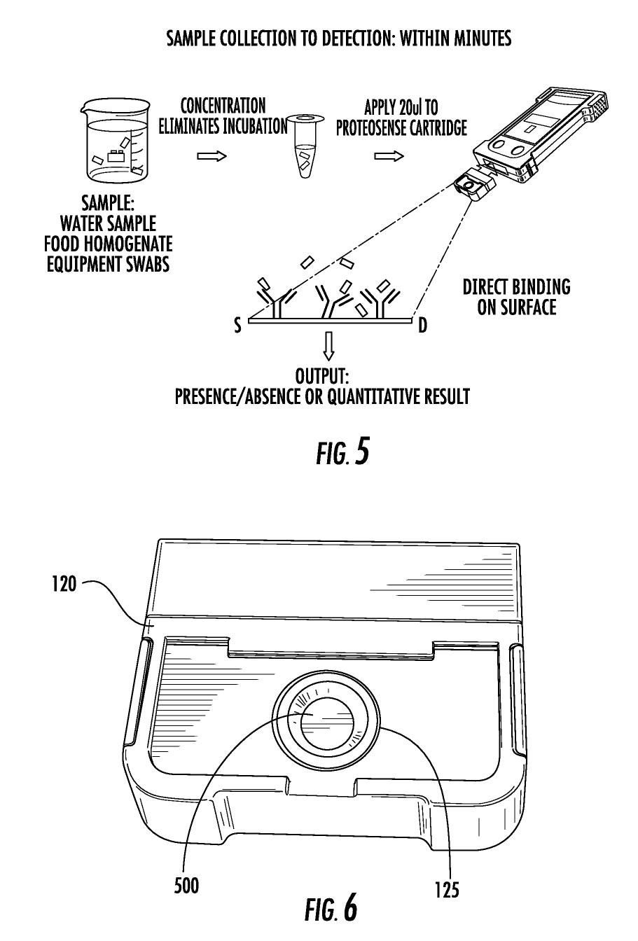

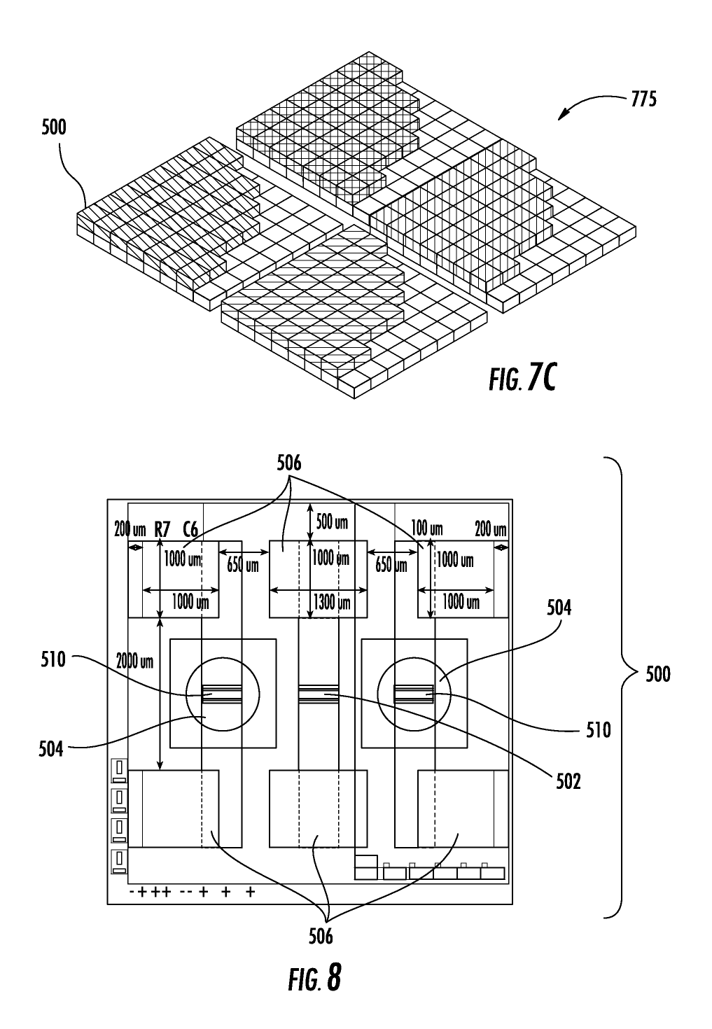

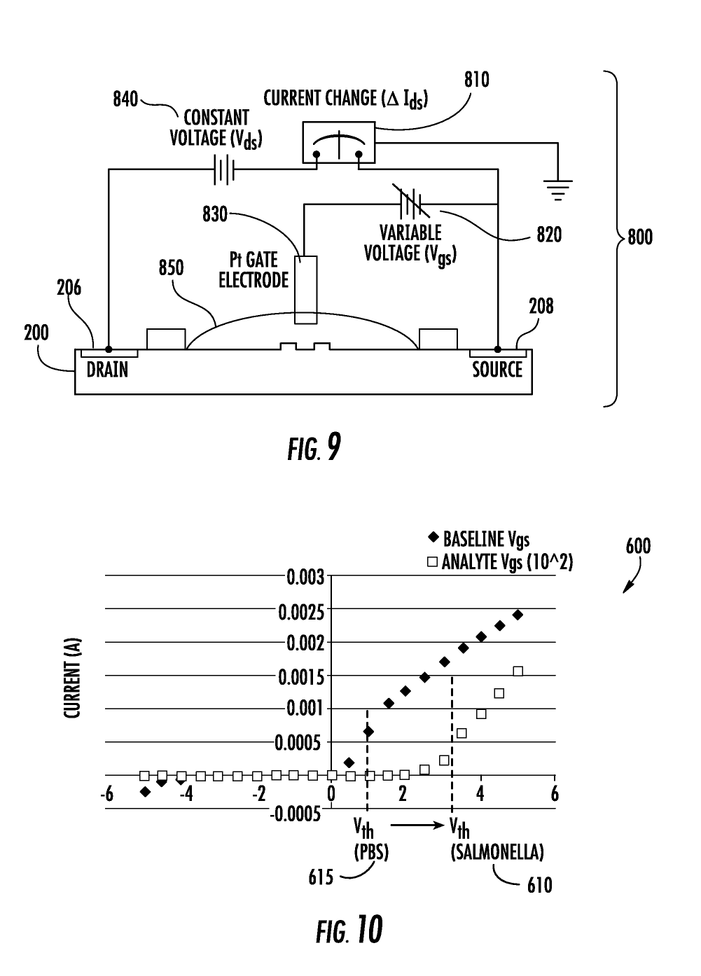

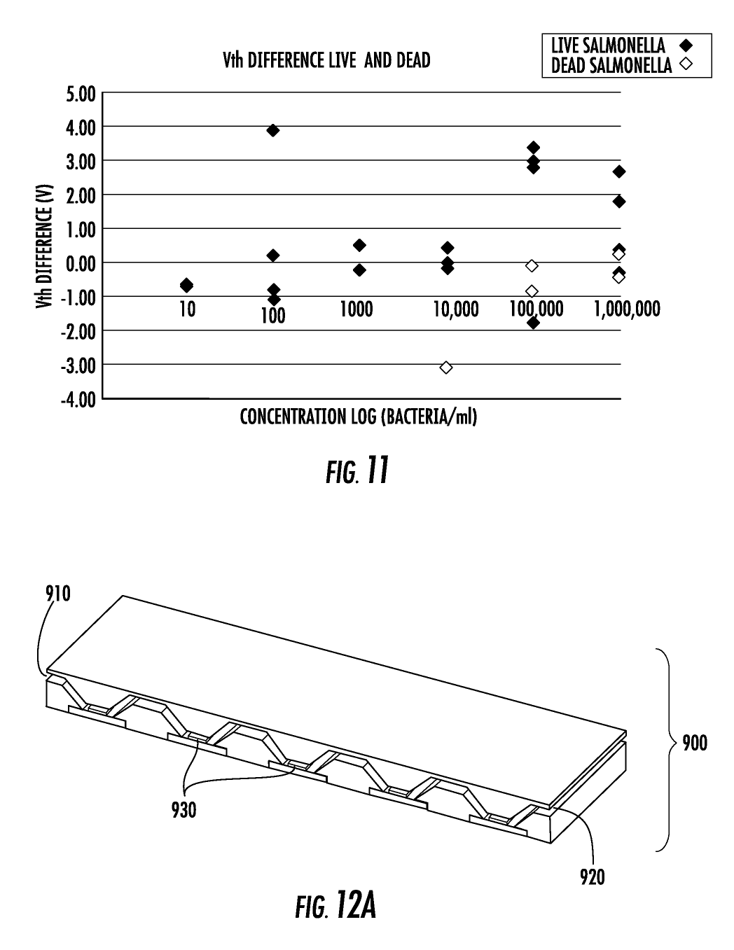

An example system (100) for the detection and quantification of analytes are illustrated in FIG. 1. Aspects of the system (100) are further detailed in the block diagram illustrated in FIG. 4. Devices and systems for detecting analytes can include a sensor cartridge (120) and a cartridge reader (110). The sensor cartridge (120) can comprise a chip (see, for example, FIG. 8) that includes an active sensor (e.g., one or more FET-based sensors configured to detect one or more analytes of interest, 510), and optionally a reference sensor (e.g., one or more reference sensors configured to provide for signal-noise improvement, 502). In certain embodiments, the sensor cartridge (120) can include two or more active sensors (e.g., at least 3, at least 5, at least 10, at least 25, at least 50, or at least 100 active sensors) disposed on the chip. When more than one active sensor is present in the chip, all the sensors can be configured to detect the same analyte of interest or different analytes of interest. Active sensors may be configured individually, in arrays (see, for example, FIGS. 7A-7C, 725, 750, and 775) or in a Whetstone bridge configuration as is well known in the art.

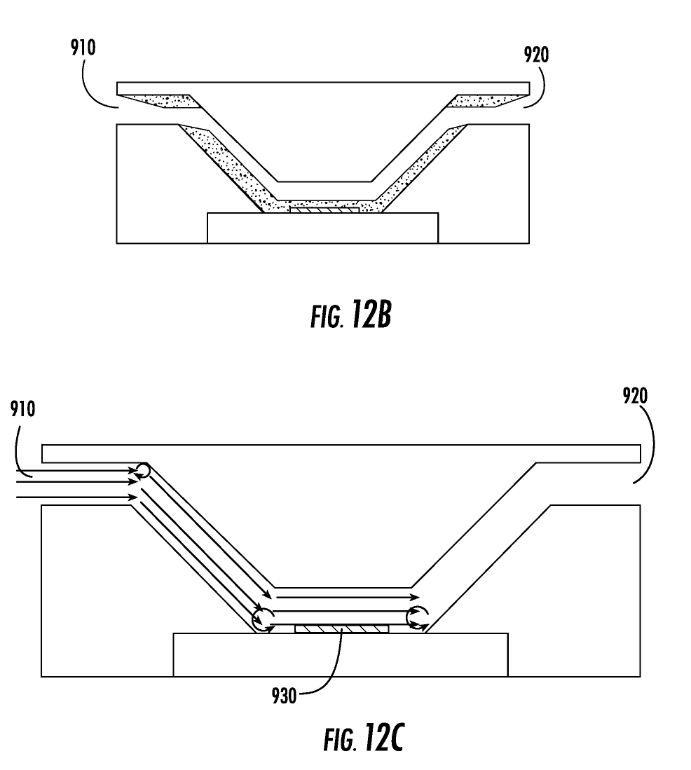

In some embodiments, the sensor cartridge (120) can further comprise a sample handling apparatus (see, for example, FIGS. 12A-12C, 900) configured to accept samples of various volumes (e.g. a sample of one analytical unit as specified in the FDA Biological Assessment Manual, typically 25 grams). The sample handling apparatus may be a flow cell configured to direct a fluid sample from a channel inlet (910) to a channel outlet (920) along a fluid flow path that induces the sample to move across one or more sensors (930) to allow the entirety of the sample volume to contact the one or more sensors in the cartridge. In some applications the sample handling apparatus may include one or more reservoirs to hold samples of various volumes (e.g. reservoirs configured to hole the sample in series with a flow cell).

The active sensor(s) in the device can comprise a substrate (e.g., sapphire); a channel disposed on the substrate, wherein the channel is substantially impermeable to ions under physiological conditions; a source electrode and a drain electrode electrically connected to the channel, wherein the source electrode and the drain electrode are formed to be separate such that the channel forms a path for current flow between the source electrode and the drain electrode; and a recognition element for the analyte of interest immobilized on the surface of the channel. The distance between the recognition element and the channel can be configured such that association of the analyte of interest with the recognition element induces a change in the electrical properties of the channel. In some applications, the dimensions of the analyte of interest may be large relative to the area of channel, causing changes to the electrical properties of the FET (e.g. a change in capacitance). The reference sensor(s) in the device can comprise a substrate; a channel disposed on the substrate, wherein the channel is substantially impermeable to ions under physiological conditions; a source electrode and a drain electrode electrically connected to the channel, wherein the source electrode and the drain electrode are formed to be separate such that the channel forms a path for current flow between the source electrode and the drain electrode; and a passivating layer disposed on the surface of the channel. The design of FET-based sensors is described in more detail below.

The cartridge reader (110) can be any suitable device configured to receive the sensor cartridge (120), and interrogate the sensors (e.g., the one or more active sensors and one or more reference sensors, when present) to detect an analyte in close proximity to the active sensors (e.g., within 50 nm of the active sensors, within 25 nm of the active sensors, within 10 nm of the active sensors, within a Debye length of the active sensors, in contact with the active sensors, and/or bound to or otherwise associated with the recognition element of the active sensors). The cartridge reader (110) can include a receiving unit (130) configured to physically receive the sensor cartridge (120), the receiving unit (130) further comprising receiving components that physically connect the sensor cartridge (120) holding it in position and receiving components that operably (e.g. electrically) connect to the active sensor and the reference sensor; and a data processing subsystem (see FIG. 4, 400) including a microcomputer (410) containing a microprocessor (405) configured to analyze an electrical property of one or more active sensors connected to a low noise preamplifier (430) and in some cases, an electrical property of the reference sensor to detect the analyte of interest. The data processing subsystem (400) can include: a microcomputer (410) containing a microprocessor (405), RAM memory (406), ROM memory (407), Flash memory (408), analog to digital converters (409) and digital to analog converters (404); an operating system (415) for control of hardware elements using a higher level programming language (e.g. C++); a barcode scanner (440); a camera (not shown); a GPS receiver (not shown) and a rechargeable battery (445). In some applications, the data processing subsystem (400) may also include a touch screen display and controller (450), interfaces for external switches (460), and one or more data communications functions (e.g. a Bluetooth module (470), a packet radio module (480), a Wi-Fi radio module (485), a USB port (490), a data storage card (495), a printer (475), and/or a cellphone connection (not shown).

The data processing subsystem (400) can include additional functions that, depending upon application, may be incorporated in either the cartridge reader (120), or the sensor cartridge (130), including: a chip assay identity module (420) which can include memory used to store assay specific information at manufacturing including but not limited to: date of manufacture, calibration data, lot number, new/used status and target analyte. This data package is implemented in a series of individual data words combined with a header and error detecting and correcting codes as is well known in the art. The new/used status word can is read by the data processing subsystem before use and modified after use to a format that prohibits reuse, functioning as a lockout.

The cartridge reader (110) can further comprise a display (150) configured to display an output from the microprocessor (405) related to the detection of the analyte of interest. The cartridge reader (110) can further comprise a communication interface configured to transmit data from the microprocessor (405) to a remote computing device wirelessly (e.g., via a GSM cellphone connection (not shown), a Wi-Fi module (485), Bluetooth module (470), or packet radio (480)) or by direct wired connection (e.g., via a USB port (490), a storage card (495), a printer (475), or an Ethernet port (not shown)).

In some embodiments, the cartridge reader (110) can further comprise an input device configured to provide user inputs to the microprocessor (405). For example, the input device can comprise a barcode reader (440), one or more input keys (140), and/or a touchscreen (150). In these embodiments, the microprocessor (405) can be configured to correlate user inputs from the input device to an electrical property of the active sensor, an electrical property of the reference sensor, an output from the microprocessor (405) related to the detection of the analyte of interest, or a combination thereof. In this way sensor readings can be correlated with samples being analyzed and analyte presence/absence and or concentration determined and communicated.

In some embodiments the cartridge reader (110) can further comprise a real-time-clock. In these embodiments, the microprocessor can be further configured to correlate sensor readings with the date and time. This way the data transmitted from the cartridge reader to a remote computing device can include the sensor readings and time of the test.

In some embodiments, the cartridge reader (110) can further comprise a global positioning receiver (GPS) (not shown). In these embodiments, the microprocessor (405) can be further configured to correlate sensor readings with the physical location of the reader (e.g. the coordinates in a field of produce). This way the data transmitted from the cartridge reader (110) to a remote computing device can include sample location in addition to the sensor readings.

In some embodiments the cartridge reader (110) can further comprise a digital camera (not shown). In these embodiments, the microprocessor (405) can be further configured to correlate sensor readings with and image of the sample or the spot where the sample was collected. This way the data transmitted from the cartridge reader (110) to a remote computing device can include the sensor readings and an image.

In some embodiments one or more sensor preamplifiers (430) may be included in the cartridge reader (110) in addition to or instead of any preamplifiers in the sensor cartridge (120).

FET-Based Sensors

Suitable FET-based sensors are described in U.S. Patent Application Publication No. 2013/0204107 to Lee et al. and U.S. Patent Application Publication No. 2013/0158378 to Berger et al., both of which are hereby incorporated by reference in their entirety.

As described above, sensor cartridges can include one or more FET-based sensors. The sensors can be used to accurately and rapidly detect and quantify analytes of interest in physiological conditions.

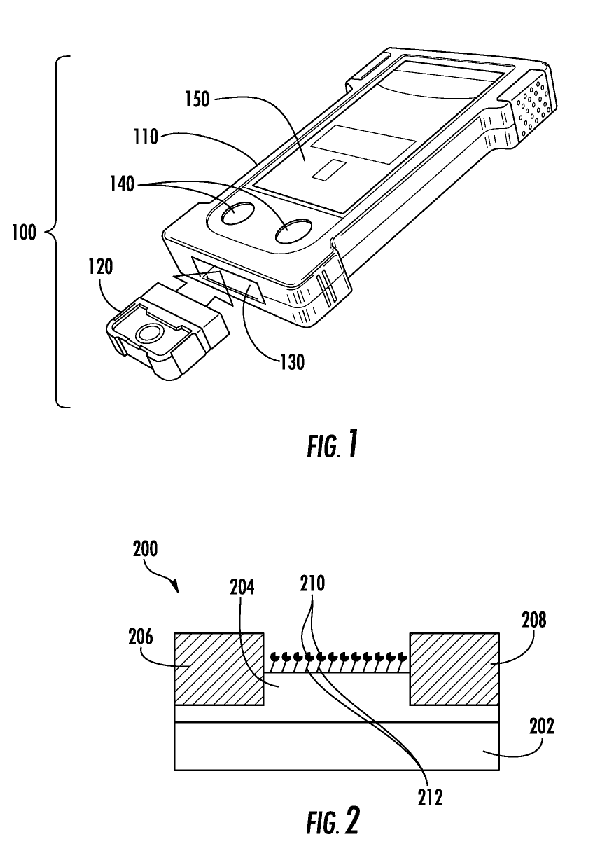

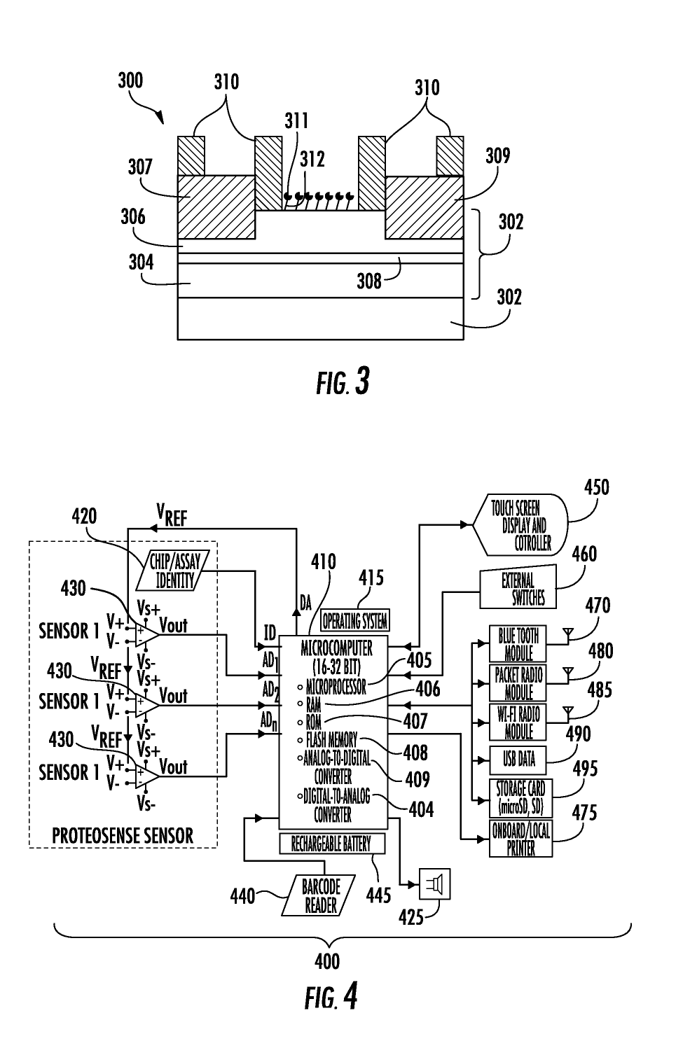

With reference to FIG. 2, a sensor (200) can comprise a substrate (202) and a channel (204) that is disposed on the substrate. The sensor can further include a source electrode (206) and a drain electrode (208) electrically connected to the channel (204). The source electrode (206) and the drain electrode (208) are formed to be separate such that the channel (204) forms a path for current flow between the source electrode and the drain electrode. In the case of active sensors, the sensor also comprises a recognition element (210) for an analyte of interest immobilized on the surface of the channel (204) via a linking group (212).

The substrate can be composed of a variety of materials which are compatible with the overall operation of the FET-based sensor. For example, the substrate may be an electric insulator (i.e., an insulating substrate) or a semiconductor coated with an insulator (i.e., an insulated semiconductor substrate) upon which one or more components of the sensor can be disposed.

Examples of suitable insulating substrates include, but are not limited to, aluminum oxide (Al₂O₃), silicon oxide, diamond, silicon nitride, calcium fluoride, glass, and combinations thereof. Examples of suitable insulated semiconductor substrates include semiconductors such as silicon carbide, silicon, aluminum nitride, gallium nitride, zinc oxide, diamond, gallium arsenide, MgZnO, titanium oxide, indium phosphide, and combinations thereof containing an insulating coating. The insulating coating can be formed from any suitable insulator, such as one or more of the insulating substrates described above. In certain embodiments, the substrate comprises Si, SiC, Al₂O₃, Group III-nitrides such as AlN or GaN, glass, diamond, or combinations thereof.

The dimensions of the substrate (e.g., length, width, and thickness) are not particularly limited, and can be selected in view of a number of criteria, including the intended application for the sensor and the size of the other sensor components (e.g., the size of the source electrode and/or drain electrode, the size of the channel, and the orientation and/or relative position of the source electrode and drain electrode).

In some embodiments, the substrate is in the form of a plate or chip. In other embodiments, the substrate may be a surface of an article, such as a medical device, probe, research instrument, vial, or microwell plate. In certain embodiments, the substrate has a thickness of at least about 10 microns (e.g., at least about 50 microns, at least about 100 microns, at least about 250 microns, or at least about 500 microns) so as to provide a sensor with sufficient mechanical strength for deployment.

Sensors further comprise a channel disposed on the substrate which forms a current path between the source electrode and the drain electrode. The channel is fabricated from one or more materials so as to be substantially impermeable to ions under physiological conditions. In some embodiments, the sensor comprises a channel fabricated from a material that is substantially impermeable to ions, such that the sensor does not exhibit significant drift in current flow over time when immersed in a physiological buffer solution (e.g., PBS buffer, pH=7.4, 150 mM Na⁺). In some embodiments, the sensor comprises a channel fabricated from a material that is substantially impermeable to ions, such that the sensor exhibits a drift in current flow of less than about 20% over a period of 10 hours when immersed in a physiological buffer solution (e.g., a drift in current flow of less than about 15% over a period of 10 hours, a drift in current flow of less than about 10% over a period of 10 hours, or a drift in current flow of less than about 5% over a period of 10 hours)

In some embodiments, the channel of the sensor comprises a Group III-nitride heterojunction. The Group III-nitride heterojunction can be formed from a first Group III-nitride layer and a second Group III-nitride layer deposited on the first Group III-nitride layer, wherein the first Group III-nitride layer and the second Group III-nitride layer have different bandgaps, such that a two-dimensional electron gas (2DEG) is generated inside the Group III-nitride heterojunction. The 2DEG can contain a very high sheet electron concentration in excess of, for example, 10¹³ carriers/cm². Group III-nitride heterojunction of this type are known in the art, and are commercially available, for example, from Cree, Inc. (Raleigh, N.C.). See also, for example, U.S. Pat. No. 5,192,987 to Khan, et al.

As used herein, the term “Group III-nitride” refers to semiconductor compounds formed from nitrogen and the elements of Group III of the Periodic Table, usually aluminum (Al), gallium (Ga) and/or indium (In). The term also refers to ternary and quaternary compounds such as AlGaN and AlInGaN. As is well understood in the art, the Group III elements can combine with nitrogen to form binary (e.g., GaN), ternary (e.g., AlGaN, AlInN) and quaternary (e.g., AlInGaN) compounds. These compounds have empirical formulas in which one mole of nitrogen is combined with a total of one mole of the Group III elements. In some embodiments, the Group III-nitride can be defined by the formula AlₓGa₁₋ₓN, where x ranges from 0 to 1.

The first Group III-nitride body can comprise, for example, a material selected from the group consisting of GaN, InN, InGaN, AlGaN, and combinations thereof. The second Group III-nitride body can comprise, for example, a material selected from the group consisting of AlGaN, AlN, InAlN, GaN, and combinations thereof. In certain embodiments, the Group III-nitride heterojunction is formed from a first Group III-nitride body that comprises GaN, and a second Group III-nitride body that comprises AlGaN.

The channel can also be formed from a semiconducting layer coated with a passivating layer that renders the channel substantially impermeable to ions under physiological conditions. For example, the channel can be formed from any of the semiconductor materials described above, such as silicon, coated with an Al₂O₃ passivating layer.

In these embodiments, the passivating layer can be a thin film of Al₂O₃ deposited on the surface of the semiconducting layer. The passivating layer can have a thickness of about 150 nm or less (e.g., about 140 nm or less, about 130 nm or less, about 120 nm or less, about 110 nm or less, about 100 nm or less, about 90 nm or less, about 80 nm or less, about 70 nm or less, about 60 nm or less, about 50 nm or less, about 40 nm or less, about 30 nm or less, or about 20 nm or less). For example, the passivating layer can have a thickness ranging from about 5 nm to about 150 nm (e.g., from about 10 nm to about 100 nm).

The source electrode and drain electrode can be fabricated from any suitable electrical conductors. Examples of suitable electrical conductors include, but are not limited to, gold, platinum, titanium, titanium carbide, tungsten, aluminum, molybdenum, chromium, tungsten silicide, tungsten nitride, and alloys and combinations thereof.

The source electrode and drain electrode, alone and in combination, can be fabricated in any suitable orientation and geometry so as to facilitate sensor operation. At least a portion of the source electrode and drain electrode are positioned in intimate contact with the channel, such that the source electrode and drain electrode are electrically connected. The source electrode and the drain electrode are formed to be separate, such that the channel (to which both the source electrode and the drain electrode are electrically connected) forms a path for current flow between the source electrode and the drain electrode.

The distance between the source electrode and the drain electrode (i.e., the length of the channel) can be selected in view of a number of factors, including the nature (e.g. the size) of the analyte being measured, the characteristics of the solution in which the analyte is being measured, and overall considerations regarding sensor design and use. In some embodiments, the distance between the source electrode and the drain electrode at their nearest point is less than 5 microns (e.g., less than 1 micron, less than 750 nm, or less than 500 nm). In other embodiments, the distance between the source electrode and the drain electrode at their nearest point is greater than 5 microns. For example, the distance between the source electrode and the drain electrode at their nearest point can range from about 0.5 microns to about 5 mm (e.g., from about 1 micron to about 1 mm; from about 5 microns to about 750 microns, from about 10 microns to about 500 microns, from about 25 microns to about 350 microns, or from about 50 microns to about 200 microns). The length of the channel can be greater than the size of the target analyte. For example, in the case of sensors designed for detecting bacterial analytes (e.g. Salmonella enterica) the length of the channel be 10-100 times greater than the size of the bacteria (e.g., 7-500 microns in length, 10-500 microns in length, 15-500 microns in length, 20-500 microns in length, 50-500 microns in length, 7-200 microns in length, 10-200 microns in length, 15-200 microns in length, 20-200 microns in length, 50-200 microns in length, 7-150 microns in length, 10-150 microns in length, 15-150 microns in length, 20-150 microns in length, 50-150 microns in length, 7-50 microns in length, 10-50 microns in length, 15-50 microns in length, 20-50 microns in length, 7-20 microns in length, 10-20 microns in length, or 15-20 microns in length).

In the case of active sensors, recognition elements can be immobilized on the channel surface via a linking group, or by direct adsorption to the channel surface. In some embodiments, the recognition elements can are immobilized on the surface of the channel via a linking group. The linking group can be selected such that the distance between the recognition element and the channel such that association of the analyte of interest with the recognition element induces a change in the electrical properties of the channel. In some cases, the linking group is selected such that the distance between the recognition element and the surface of the channel is less than about 10 nm (e.g., less than about 9 nm, less than about 8 nm, less than about 7 nm, less than about 6 nm, less than about 5 nm, less than about 4 nm, less than about 3 nm, less than about 2 nm, or less than about 1 nm).

In some embodiments, the linking group comprises a polyvalent linking group. Polyvalent linking groups are derived from polyvalent linkers (i.e., linkers which associate with the channel surface via two or more chemical moieties and have the capacity to be covalently or non-covalently linked to a recognition element). For example, the polyvalent linking group can be derived from a small molecule linker that forms two or more covalent bonds with the channel surface and a covalent bond with the recognition element.

In some embodiments where the linking group comprises a polyvalent linking group, the recognition element is bound to an interfacial polymeric film, such as a silane polymer film derived from trialkoxysilane monomers. In principle, any polymer producing an interfacial film of suitable thickness (e.g., less than 10 nm, less than about 9 nm, less than about 8 nm, less than about 7 nm, less than about 6 nm, less than about 5 nm, less than about 4 nm, less than about 3 nm, less than about 2 nm, or less than about 1 nm) and with capacity to be linked to recognition elements (covalently or non-covalently) can serve as a polyvalent linking group. Examples of suitable polyvalent linking groups include thin films derived from polyvalent linkers including 3-aminopropyl)triethoxysilane (APTES), (3-glycidyloxypropyl)trimethoxysilane, (3-mercaptopropyl) trimethoxysilane, vinyltrimethoxysilane, allyltrimethoxysilane, (3-bromopropyl) trimethoxysilane, triethoxyvinylsilane, triethoxysilane aldehyde (TEA), and combinations thereof.

In certain embodiments, the linking group comprises a monovalent linking group. Monovalent linking groups are derived from monovalent linkers (i.e., linkers which associate with the channel surface via a single chemical moiety and have the capacity to be covalently or non-covalently linked to a recognition element). For example, monovalent linking groups can possess a first moiety which is associated with or bound to the channel surface, and a second moiety which is associated with or bound to the recognition element. In this way, the monovalent linker forms a molecular monolayer which tethers the recognition element to the channel surface.

The monvalent linking group can be derived from a heterobifunctional small molecule which contains a first reactive moiety and a second reactive moiety. The first reactive moiety can be reactive with the channel surface (e.g., with the Group III-nitride heterojunction) and the second reactive moiety can be reactive with one or more moieties present in the recognition element. In some embodiments, the monvalent linking group comprises an alkyl group having from 1 to 6 carbon atoms in its backbone.

In some embodiments, the monovalent linking group is derived from a linker which comprises a monoalkoxysilane moiety. In some embodiments, the monovalent linking group is derived from a linker which comprises a monohalosilane moiety. Examples of suitable monovalent linkers include (3-aminopropyl) dimethylethoxysilane (APDMES), (3-glycidoxypropyl) dimethylethoxysilane, (4-chlorobutyl)dimethylchlorosilane, and combinations thereof.

In the case of active sensors, the sensors further include a recognition element for an analyte of interest immobilized in proximity to the channel surface (e.g., immobilized on the surface of the channel), such that association of the analyte of interest with the recognition element induces a change in the electrical properties of the sensor (e.g., an electrical property of the channel). Recognition elements for particular analytes of interest are known in the art, and can be selected in view of a number of considerations including analyte identity, analyte concentration, and the nature of the sample in which the analyte is to be detected. Suitable recognition element include antibodies, antibody fragments, antibody mimetics (e.g., engineered affinity ligands such as AFFIBODY® affinity ligands), peptides (natural or modified peptides), proteins (e.g., recombinant proteins, host proteins), oligonucleotides, DNA, RNA (e.g., microRNAs), aptamers (nucleic acid or peptide), and organic small molecules (e.g., haptens or enzymatic co-factors).

In some embodiments, the recognition element selectively associates with the analyte of interest. The term “selectively associates”, as used herein when referring to a recognition element, refers to a binding reaction which is determinative for the analyte of interest in a heterogeneous population of other similar compounds. Generally, the interaction is dependent upon the presence of a particular structure (e.g., an antigenic determinant or epitope) on the binding partner. By way of example, an antibody or antibody fragment selectively associates to its particular target (e.g., an antibody specifically binds to an antigen) but it does not bind in a significant amount to other proteins present in the sample or to other proteins to which the antibody may come in contact in an organism.

In some embodiments, a recognition element that “specifically binds” an analyte of interest has an affinity constant (Kₐ) greater than about 10⁵ M⁻¹ (e.g., greater than about 10⁶ M⁻¹, greater than about 10⁷ M⁻¹, greater than about 10⁸ M⁻¹, greater than about 10⁹ M⁻¹, greater than about 10¹⁰ M⁻¹, greater than about 10¹¹ M⁻¹, greater than about 10¹² M⁻¹, or more) with that analyte of interest.

In certain embodiments, the recognition element comprises an antibody. The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen. The term encompasses intact and/or full length immunoglobulins of types IgA, IgG (e.g., IgG1, IgG2, IgG3, IgG4), IgE, IgD, IgM, IgY, antigen-binding fragments and/or single chains of complete immunoglobulins (e.g., single chain antibodies, Fab fragments, F(ab′)2 fragments, Fd fragments, scFv (single-chain variable), and single-domain antibody (sdAb) fragments), and other proteins that include at least one antigen-binding immunoglobulin variable region, e.g., a protein that comprises an immunoglobulin variable region, e.g., a heavy (H) chain variable region (VH) and optionally a light (L) chain variable region (VL). The light chains of an antibody may be of type kappa or lambda.

An antibody may be polyclonal or monoclonal. A polyclonal antibody contains immunoglobulin molecules that differ in sequence of their complementarity determining regions (CDRs) and, therefore, typically recognize different epitopes of an antigen. Often a polyclonal antibody is derived from multiple different B cell lines each producing an antibody with a different specificity. A polyclonal antibody may be composed largely of several subpopulations of antibodies, each of which is derived from an individual B cell line. A monoclonal antibody is composed of individual immunoglobulin molecules that comprise CDRs with the same sequence, and, therefore, recognize the same epitope (i.e., the antibody is monospecific). Often a monoclonal antibody is derived from a single B cell line or hybridoma. An antibody may be a “humanized” antibody in which for example, a variable domain of rodent origin is fused to a constant domain of human origin or in which some or all of the complementarity-determining region amino acids often along with one or more framework amino acids are “grafted” from a rodent, e.g., murine, antibody to a human antibody, thus retaining the specificity of the rodent antibody.

In certain embodiments, the recognition element comprises an immunoglobulin G (IgG) antibody, a single-chain variable fragment (scFv), or a single-domain antibody (sdAb).

In certain embodiments, the recognition element comprises a receptor, such as a soluble receptor, for use in detecting ligands of the receptor as the analyte of interest.

In some embodiments, the recognition element comprises an antigen or antigenic hapten. In certain embodiments, the antigenic hapten is not biotin or a derivative thereof. Any suitable antigen can be used. For example, the antigen can be viral antigens, bacterial antigens, tumor antigens, tissue specific antigens, fungal antigens, parasitic antigens, human antigens, botantical antigens, non-human animal antigens, allergens, synthetic antigens, or combination thereof.

In certain embodiments, the recognition element is a recognition element for one or more food-borne pathogens, such as Salmonella enterica, E. coli, or Listeria monocytogenes. For example, the recognition element can be an antibody or combination of antibodies that selectively associates with Salmonella enterica, E. coli, or Listeria monocytogenes.

The sensors described herein can further contain one or more additional components. For example, sensors can further comprise an insulator disposed on the source electrode, the drain electrode, or combinations thereof. The insulator can be configured to permit a conductive fluid to be applied to the surface of the channel without the conductive fluid completing a short circuit between the source electrode and the drain electrode. Insulators can also be disposed on a portion of the channel surface, for example, to create a well into which fluid samples can be applied.

Sensors can further include a gate electrode configured to apply a gate bias to the channel. A gate bias can be applied to the channel to allow the sensor to operate in the subthreshold regime. This can allow the sensor to be more sensitive to interaction of the recognition element with the analyte of interest. In some embodiments, the sensor is back-gated (i.e., it includes a gate electrode beneath the channel, such as within the substrate, which is configured to apply a gate bias to the channel). The sensor can include a side gate positioned adjacent to the channel, and configured to apply a gate bias to the channel. In some embodiments, a floating electrode in contact with the fluid in which the sensor is immersed is used to apply the gate bias.

The sensor can further include electronic circuitry configured to detect a change in an electrical property of the channel. For example, the sensor can include electronic circuitry configured to measure a change in current flow, a change in voltage, a change in impedance, or combinations thereof.

An exemplary FET-based active sensor is illustrated in FIG. 3. The sensor (300) comprises a substrate (302) and a channel comprising a Group III-nitride heterojunction (302) disposed on the substrate. The Group III-nitride heterojunction (302) comprises a first Group III-nitride layer (304) and a second Group III-nitride layer (306). The first Group III-nitride layer (304) and the second Group III-nitride layer (306) have different bandgaps, such that a two-dimensional electron gas (308) is generated inside the Group III-nitride heterojunction (302). The sensor further includes a source electrode (307) and a drain electrode (309) electrically connected to the Group III-nitride heterojunction (302). The source electrode (307) and the drain electrode (309) are formed to be separate such that the Group III-nitride heterojunction (302) forms a path for current flow between the source electrode (307) and the drain electrode (309). The sensor also includes a recognition element (311) for an analyte of interest immobilized on the surface of the Group III-nitride heterojunction (302) via a linking group (312). An insulator (310) is disposed on the source electrode (307), the drain electrode (309) and the Group III-nitride heterojunction (302) to permit a conductive fluid to be applied to the surface of the Group III-nitride heterojunction (302) without the conductive fluid completing a circuit between the source electrode (307) and the drain electrode (309).

The active sensors described herein can be used to rapidly and accurately detect an analyte in physiological conditions. As used herein, the term “physiological conditions” refers to temperature, pH, ions, ionic strength, viscosity, and like biochemical parameters which exist extracellularly or intracellularly in an organism. In some embodiments, the physiological condition refers to conditions found in serum and/or blood of an organism. In some embodiments, the physiological condition refers conditions found in a cell in an organism. In some embodiments, physiologic condition refers to conditions found in a homogenous or heterogeneous solution derived from plant and/or animal origin.

Particular in vitro conditions to mimic physiological conditions can be selected by the practitioner according to conventional methods. For general guidance, the following buffered aqueous conditions can be applicable: 10-250 mM NaCl, 5-50 mM Tris HCl, pH 5-8, with optional addition of divalent cation(s) and/or metal chelators and/or nonionic detergents and/or membrane fractions and/or antifoam agents and/or scintillants. In general, in vitro conditions that mimic physiological conditions comprise 50-200 mM NaCl or KCl, pH 6.5-8.5, 20-45° C., and 0.001-10 mM divalent cation (e.g., Mg²⁺, Ca²⁺); preferably about 150 mM NaCl or KCl, pH 7.2-7.6, 5 mM divalent cation.

The active sensors can be used to detect an analyte of interest by contacting the analyte of interest with the sensor, and measuring a change in an electrical property of the sensor channel. The change in electrical property can be, for example, a change in current flow, a change in voltage, a change in impedance, or combinations thereof.

In some cases, the methods can further include applying a gate bias to the channel. The gate bias can be applied using a gate electrode positioned beneath the channel (i.e., a back gate), adjacent to the channel (e.g., a side gate), or in contact with a conductive fluid contacting the channel surface (e.g., a floating electrode). The gate bias can be selected to allow the sensor to operate in the subthreshold regime. This can allow the sensor to be more sensitive to interaction of the recognition element with the analyte of interest.

The methods described herein can be used to detect analytes in solution. In some embodiments, the analyte of interest is present in an aqueous solution.

The analyte of interest can be present in a biological sample. “Biological sample,” as used herein, refers to a sample obtained from or within a biological subject, including samples of biological tissue or fluid origin obtained in vivo or in vitro. Such samples can be, but are not limited to, bodily fluid, organs, tissues (e.g., including resected tissue), fractions and cells isolated from mammals including, humans. Biological samples also may include sections of the biological sample including tissues (e.g., sectional portions of an organ or tissue). The term “biological sample” also includes lysates, homogenates, and extracts of biological samples.

In certain embodiments, the analyte of interest is present in a bodily fluid. “Bodily fluid”, as used herein, refers to a fluid composition obtained from or located within a human or animal subject. Bodily fluids include, but are not limited to, urine, whole blood, blood plasma, serum, tears, semen, saliva, sputum, exhaled breath, nasal secretions, pharyngeal exudates, bronchoalveolar lavage, tracheal aspirations, interstitial fluid, lymph fluid, meningal fluid, amniotic fluid, glandular fluid, feces, perspiration, mucous, vaginal or urethral secretion, cerebrospinal fluid, and transdermal exudate. Bodily fluid also includes experimentally separated fractions of all of the preceding solutions, as well as mixtures containing homogenized solid material, such as feces, tissues, and biopsy samples.

The methods described herein can be used to detect an analyte of interest ex vivo. In these instances, methods for detecting an analyte of interest can include collecting a biological sample from a patient, contacting the analyte of interest in the biological with a sensor, and measuring a change in an electrical property of the sensor channel. In certain embodiments, the ex vivo sample is a biological fluid, lysate, homogenate, or extract.

The methods described herein can be used to detect an analyte of interest in vitro (i.e., the analyte of interest is contacted with the sensor in vitro). Such methods can be used, for example, to monitor tissue cultures.

The analyte of interest can be present in an environmental sample, such as a water sample, air, soil leachate, or environmental test swabs (e.g. Enviro Swab, 3M, St Paul, Minn.).

The methods can be used to determine a presence of the analyte of interest, to determine the concentration of the analyte of interest, or a combination thereof.

The active sensors and methods described herein can be used to detect a variety of analytes. In order to be detected by the FET-based sensor, the analyte of interest must generate an electric field in proximity to the channel surface. In some cases, the analyte is charged (e.g., the analyte has a net negative or a net positive charge). In other embodiments, the analyte of interest has a net neutral charge, but contains one or more charged regions such that when associated with the recognition element, an electric field is generated which modulates the electrical properties of the channel.

The analyte of interest can comprise a macromolecule, such as a biomacromolecule. “Macromolecule,” as used herein, refers to a large molecule, typically having a high relative molecular weight, such as a polymer, polysaccharide, protein, peptide, or nucleic acid. The macromolecule can be naturally occurring (i.e., a biomacromolecule) or can be prepared synthetically or semi-synthetically. In certain embodiments, macromolecules have a molecular weight of greater than about 1000 amu (e.g., greater than about 1500 amu, or greater than about 2000 amu).

In some embodiments, the analyte of interest is an antibody, peptide (natural, modified, or chemically synthesized), protein (e.g., glycoproteins, lipoproteins, or recombinant proteins), polynucleotide (e.g, DNA or RNA), lipid, polysaccharide, pathogen (e.g., bacteria, virus, or fungi, or protozoa), or a combination thereof. In certain embodiments, the analyte of interest comprises a biomarker for a disease process in a patient.

Reference sensors are similar to those above; however, they lack the ability to interact with the analyte of interest. For example, a reference sensor can comprise a substrate and a channel that is disposed on the substrate. The sensor can further include a source electrode and a drain electrode electrically connected to the channel. The source electrode and the drain electrode are formed to be separate such that the channel forms a path for current flow between the source electrode and the drain electrode. In the case of reference sensors, the sensor can also comprises a passivating layer on the surface of the channel that insulates the channel from contact with the sample solution.

Alternative Sensors

Other sensor technologies having suitable sensitivity, specificity and limit of detection may be used instead of FET sensors in the devices and systems described herein. For example, electrochemical sensors, such as the electrochemical sensors described in U.S. Pat. No. 8,585,879 to Yau, et al., which is hereby incorporated by reference in its entirety, can be used instead of FET sensors in the devices and systems described herein. When using an electrochemical sensor such as those described in U.S. Pat. No. 8,585,879 to Yau, et al., the reader circuit would be modified to include a potentiostat, as is well known in the art.

Another sensor that could be used in the devices and systems described herein is found in United States Patent Application Publication No. 2013/0249574 to House, which is hereby incorporated by reference in its entirety. The sensor described herein is an antibody treated nanotube array. Configurations of the array to achieve high sensitivity and specificity could require changes to the reader circuit, as described in the reference publication.

These are only two examples of alternative biosensors in the art that could be incorporated in the devices and systems described herein. One skilled in the art would appreciate that additional sensors could be used in the systems and devices described herein.

Methods of Use

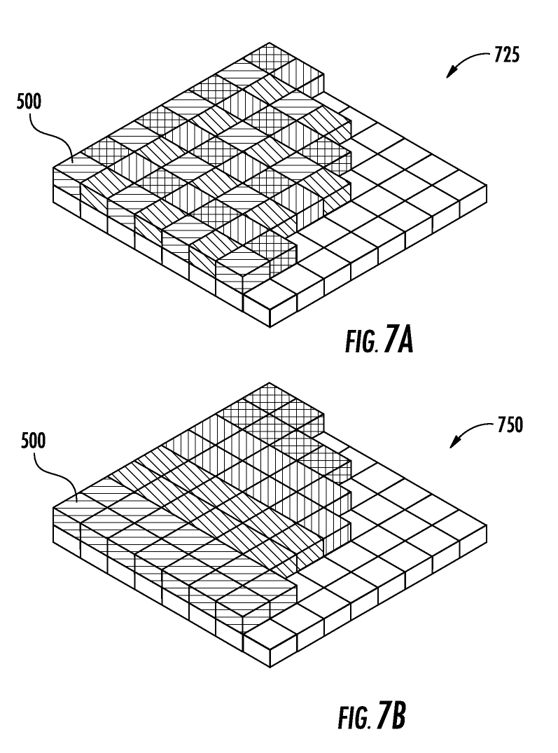

Methods of using the devices and systems described herein are schematically illustrated in FIG. 5. Methods can involve contacting the active sensors on the chip of the sensor cartridge with a sample solution, engaging the sensor cartridge into the cartridge reader, measuring a change in an electrical property of the sensor. The change in electrical property can be, for example, a change in current flow, a change in voltage, a change in impedance, a change in capacitance, or combinations thereof. Methods can further involve correcting the measured change using the reference sensor, and processing the measured change and/or corrected change to obtain additional information, such as analyte concentration.

The devices and systems described herein can be used in place of existing immunoassays, such as ELISAs, in clinical and research settings to detect proteins and peptides and/or to measure the concentration of proteins and peptides. For example, the devices and systems described herein can be used to detect antibodies or antigens in a sample.

The devices and systems described herein can be used in clinical and healthcare settings to detect biomarkers (i.e., molecular indicators associated with a particular pathological or physiological state). The devices and systems described herein can be used to diagnose infections in a patient (e.g., by measuring serum antibody concentrations or detecting antigens). For example, the devices and systems described herein can be used to diagnose viral infections (e.g., Ebola, HIV, hepatitis B, hepatitis C, rotavirus, influenza, or West Nile Virus), bacterial infections (e.g., E. coli, Lyme disease, or H. pylori), and parasitic infections (e.g., toxoplasmosis, Chagas disease, or malaria). The devices and systems described herein can be used to rapidly screen donated blood for evidence of viral contamination by HIV, hepatitis C, hepatitis B, and HTLV-1 and -2. The devices and systems described herein can also be used to measure hormone levels. For example, the sensors can be used to measure levels of human chorionic gonadotropin (hCG) (as a test for pregnancy), Luteinizing Hormone (LH) (to determine the time of ovulation), or Thyroid Stimulating Hormone (TSH) (to assess thyroid function). The devices and systems described herein can be used to diagnose or monitor diabetes in a patient, for example, by measuring levels of glycosylated hemoglobin, insulin, or combinations thereof. The devices and systems described herein can be used to detect protein modifications (e.g., based on a differential charge between the native and modified protein and/or by utilizing recognition elements specific for either the native or modified protein).

The devices and systems described herein described herein can also be used, for example, to detect and/or monitor the levels of therapeutic peptides in vivo. For example, the devices and systems described herein can be used to detect and/or monitor the levels of growth hormone, interferon-alpha, rituximab, infliximab, etanercept, or bevacizumab in vivo. This could be used during treatment (e.g., to titrate clinically preferred levels of a therapeutic peptide) as well as during clinical trials.

The devices and systems described herein can be used to detect proteinaceous toxins, including mycotoxins, venoms, bacterial endotoxins and exotoxins, and cyanotoxins. For example, the devices and systems described herein could be used to detect botulinum toxin, ricin, tetanus toxin, C. difficile toxin A, C. difficile toxin B, or staphylococcal enterotoxin B (SEB).

The devices and systems described herein can also be used in other commercial applications. For example, the devices and systems described herein can be used in the food industry to detect potential food allergens, such as wheat, milk, peanuts, walnuts, almonds, and eggs. The devices and systems described herein can be used to detect and/or measure the levels of proteins of interest in foods, cosmetics, nutraceuticals, pharmaceuticals, and other consumer products.

In some embodiments, the devices and systems described herein can be used to detect food-borne pathogens, such as Salmonella enterica, E. coli, or Listeria monocytogenes in food and environmental samples

The devices and systems described herein can be used in the biotechnology industry to measure the concentration of biomolecules, such as antibodies, during manufacture.

The devices and systems described herein can be used in process control applications, for example to continually monitor food, wash water, or other samples for food-borne pathogens such as Salmonella enterica, E. coli, or Listeria monocytogenes.

By way of non-limiting illustration, examples of certain embodiments of the present disclosure are given below.

### EXAMPLES

### Example 1: Handheld Sensor for the Detection and Quantification of Salmonella

Salmonella bacteria is a significant public health concern. It is estimated that Salmonella enterica is responsible for approximately 1 million infections/year in the U.S. alone. Salmonella is primarily transmitted through contaminated food sources. The accurate detection of Salmonella during harvest, food processing, manufacturing and shipping are critical to preventing the spread of Salmonella. However, existing diagnostics (PCR, ELISA) are time consuming (>48 hrs to result) and require long incubation times (>12 hrs). Improved rapid devices, systems, and methods for the detection and quantification of analytes, such as Salmonella, offer the possibility to reduce the spread of food-borne illnesses.

Introduction to Proposed Handheld ProteoSense Salmonella ent. Detector

The ProteoSense Salmonella enterica detector is a device for the rapid detection of analytes based on the binding of analytes to recognition elements (e.g., antibodies) present at the surface of a heterogeneous junction field effect transistor (FET). ProteoSense sensors aim to reduce the time and effort associated with Salmonella ent. testing for interested parties at all stages of produce production.

The ProteoSense technology is packaged in a convenient, lightweight hand reader. Chips are produced from an AlGaN wafer processed using standard micro/nanofabrication techniques.

By using a signal associated with analyte binding, the ProteoSense Sensor and Reader reduce detection times to minutes. These sensors do not require long, if any, incubation times as with conventional tests. Typical times to detection are on the order of minutes (e.g., 5-15 minutes). The detection times are reduced by relying solely on the time-to-binding of the affinity elements (e.g., antibodies) and the target molecules. This decrease in time results in produce that can be tested prior to shipment, reducing the rate of recalls.

ProteoSense Sensor Advantages

The immunoFETs produced by ProteoSense offer solutions to major hurdles that have been experienced by previous bio and immunoFET devices. Previous analysis suggested that the use of antibodies would be incapable of producing sufficient gate charge flow to provide detectable current change. However, due to the flexibility and binding orientation of antibodies target molecules are able to bind and alter the gate charge to allow signal shifts.

Further enhancing the ProteoSense sensors capacities is the implementation of an optimized silanization layer. The silane group provides a link between the hydoxylized surface of the FET and a carboxyl group on the antibody. In the past devices attempt functionalization using a standard silane which has a trivalent nature. The trivalent nature of the silane requires that great care be taken with the surface functionalization as to prevent a potentially infinite polymerization of the surface. As distance between the bound biomolecule and the FET increase, sensitivity decreases in both magnitude and reliability.

Additionally, the immunoFETs are based on an AlGaN/GaN HFET sensors. The AlGaN substrate makes the FET significantly less susceptible to ions from physiological solutions leading to greater signal stability as well as greater signal to noise ratio. This AlGaN system is further enhanced by the deposition of a gallium nitride protective cap which is oxidized to provide a binding site for hydroxylation and further functionalization thereby minimizing the distance between the FET gate and the biomolecule.

Proposed ProteoSense Handheld Unit

The immunoFET sensors are packaged in a removable sensor cartridge containing one or more sensors configured to detect one or more analytes and are measured with a re-useable handheld reader. For illustration, a cartridge may contain one or more semiconductor dies each containing one or more immunoFETs. Each immunoFET may be configured to test the same or different targets in a sample. This allows for one test to cover one or several targets.

The handheld reader which is designed to manage calibration, data collection, interpretation, storage, and communication in a single user-friendly device (e.g. an Android, Windows or iOS smartphone with appropriate sensor interface circuits and application software). The handheld device includes a touch screen and optional external buttons or switches which are then connected to a microcomputer containing a device operating system. The device can be connected in a wired method for example USB cable, or a wireless method, for example Bluetooth, Packet Radio, Wireless Network, or cellular radio. The device can also contain a speaker for audible confirmations or alarms. The device can also include a bar code or QR scanner capability to be able to detect which cartridge is being used, to monitor samples repeatedly or import sample descriptions/identifiers. In these cases, the device can include the capability to input information about a sample contemporaneous with the measurement of the sensor cartridge associated with that sample, allowing the readings from a sensor cartridge to be efficiently correlated with information regarding the sample that is measured.

The ProteoSense handheld reader is designed with all users in mind. Signal analysis can be performed by the software, providing for raw data outputs and/or interpretations of that data associated with calibration and characterization data from internal company tests indicating a positive presence, negative presence, and options to report or store data by sample descriptions. These reports can be accompanied by audible warnings or automatically report via wireless networking. The device can also include an optional or built in printer associating antigen levels with sample descriptions.

When placed in the reader, the sensor cartridge can identify itself to the reader and run a self-check prior to testing to guarantee accurate results. The on board computer can be configured to automatically load the appropriate parameters which have been determined through characterization and testing prior to interrogating FET-based sensor on the chip in the sensor cartridge. Following calibration, the reader can be configured to prompt the user for input to begin the test.

In some embodiments, the cartridge reader can further comprise a global positioning receiver (GPS). In these embodiments, the microprocessor can be further configured to correlate sensor readings with the physical location of the reader (e.g. the coordinates in a field of produce). This way the data transmitted from the cartridge reader to a remote computing device can include sample location and sensor readings.

An alternative configuration of the reader can be constructed by using the hardware and software of a smartphone such as an iPhone, Windows, or Android-based phone. In this case the phone would interface with cartridges through the use of an i/o port such as the Thunderbolt port found on some iPhones or any of the existing ports on a smartphone. The software to run the sensor calibration, measurement and reporting processes can be engineered as an application to run on the phone's native operating system.

ProteoSense Sensor Cartridge

The heart of the ProteoSense technology is the modular cartridge containing one or more the AlGaN immunoFETs. The one or more immunoFETs disposed on the chip in the sensor cartridge can be pre-functionalized with recognition elements (e.g., antibodies) for an analyte of interest prior to delivery to an end user.

The chip can include a single or combination assay (e.g., sensors for one or more analytes of interest). Optionally, the one or more sensors can be disposed on the chip in a functional geometry. Single FET designs can utilize one or more FETs. Designs incorporating FETs functionalized to more than one target can be arranged with one or more sensors to each target, as discussed in more detail below.

FIG. 6 shows a rendering of an example sample cartridge. The cartridge can be designed ergonomically to allow for ease of placement into the handheld reader as well as removal of cartridges after use. Examples of these could be recessed textured regions for improving grip. Grips can be located remotely from the sensor surface and inlet to avoid accidental contamination during device loading.

The cartridge can be packaged with a seal over the inlet port to prevent external contamination from fouling the sensor prior to use or to enclose a buffer solution in contact with the functionalized sensor surfaces to prevent deterioration of the antibodies. The seal can be removed by the user prior to contacting the sensor cartridge with a sample. When packaged with a buffer solution, the cartridge itself can be sealed to prevent leakage.

The cartridge can also be of a shape or connector configuration that prevents incorrect insertion of the cartridge into the sensor. This geometry can also act as a visual indication of cartridge orientation. An example might be a rounded edge furthest from the connection and a square edge at the connecting edge of the cartridge.

The cartridge can also include aesthetic features. An example of this could be a ProteoSense company logo. Another example could be a color band distinguishing assays or adhering to company color schemes. Another example could be instructional logos. This could include statements like “This side up”, arrows, or usage diagrams.

The cartridge can include one or more electrical connections to establish power and signal connection between the immunoFET sensors on the chip and the reader.

The cartridge can include an inlet for a sample volume of fluid. Electrical connections can be positioned away from this inlet to prevent unintentional shorting during connection to the handheld sensor. The cartridge can be configured to connect to the hand held reader in a secure manner. An example of this connection could be a snap-fit compression fit connection. Another example could be a standard compression fit modular electrical connection or port.

The cartridge inlet can include a port which brings fluid samples into contact with the sensor surface. This could be a rectangular capillary channel or a simple opening above the sensor surface. Another example could be a microfluidic configuration driven by mechanical pressure, electro-osmotic pressure, or capillary force, as shown in FIGS. 12A, 12B, and 13C. Sensor inlet may be protected after functionalization through the use of packaging which can be removed from the inlet source immediately before use but after cartridge is placed in the handheld reader. Alternatively, the inlet may utilize a geometry that prevents contamination during cartridge loading.

Cartridges with more than one sensors can include an array of sensors arranged in a geometric pattern. The geometry should provide for representative population of the introduced solution. One example of an assay with three targeted antigens could be based off of a geometric pattern of FET placement aligned with a grid as shown in FIGS. 7A-7C. An additional geometric pattern could be radial or circular. Another example could be a hexagonal arrangement of sensors. Another configuration could be alternating linear regions of a number of discrete functionalized regions.

One embodiment of a chip for use in sensor cartridges is illustrated in FIG. 8. The chip (500) can include two active sensors (e.g., two FET-based sensors 510 configured to detect an analyte such as Salmonella), one reference sensor (e.g., a FET-based sensor 502 that is buried under a passivation layer so as to not contact a sample solution), and electrical contacts (506) that are configured to electrically connect the sensors on the chip with external electrical components used to measure the electrical properties of the sensor channels. Wells (504) can be disposed around the active sensors to retain fluid samples in proximity to the sensor during analysis.

The sensor cartridge can contain either a direct connection to the microcontroller for each sensor or utilizer a signal multiplexer in the case of larger devices. It may also contain one or more preamplifiers to increase signal level and improve noise reduction. The cartridge can also contain a connection that will provide specific assay information to the microcontroller as to pass the identity and setting for the chip to the handheld reader. This information can be stored in nonvolatile memory in the cartridge or through other techniques and structures known in the art. This information could also be transferred via a barcode.

Cartridges with multiple sensors included would, in theory, present an opportunity to provide analysis with a built-in redundancy. Single target cartridges would be capable of providing increased quantitative information through the use of raw data or using a computational algorithm to compute total quantity. Cartridges providing a combination assay would be capable of a similar computation but with reduced sensitivity that would likely decrease as the number of sensors to a specific target are decreased. Sensor density would be a function of the practicality of functionalizing each discrete sensor to a given antibody.

Multiple sensors could be configured in several ways. One example would be functionalizing one or more HFET devices to one antibody and then incorporating these HFETs into an array of other HFETs which are functionalized to different antibodies as shown in FIG. 7B. These devices would be mounted to the packaging in close proximity or directly in contact with one another. Alternatively they could be mounted directly to one another or another substrate and then to the packaging. Another example could be functionalizing individual devices according to a geometry as previously mentioned.

The number of total sensors is related directly to the density which can be accommodated by functionalization and independent wiring. For example, functionalization might occur through the use of a capillary pipette which addresses individual sensors in a pattern. If the pitch (center-to-center) distance of the capillaries is larger than the pitch of a sensor unit wired and packaged, then the pitch of the functionalization system becomes the limiting factor in the density of the sensors. Likewise if a unit sensor including wiring, passivation layers, has the largest pitch then this becomes the limiting dimension for the density of sensors.

If the functionalization system is the limiting factor, for instance the ability to address individual fluidic volumes is limited to a minimum pitch then this pitch (or a larger one to accommodate an integer of sensors) could be used to define a unit sensor wherein there are multiple FETs functionalized to the same analyte target. An example would be if the unit sensor was 25 microns squared and the minimum pitch of the functionalization mechanism is 200 microns (in orthogonal directions) with a 100×100 micron functionalization then the unit would be able to be contain 16 unit sensors all functionalized to the same analyte target.

Chip Fabrication and Testing

Testing and characterization of sensors can be performed on an integrated connection platform that is designed for the chips die as they are currently made. This does not represent the final form of the chip and meter but rather a developer kit to interact with the chip that is analogous to the final reader.

The fabrication/functionalization of the chips can be performed in a standard laboratory environment. Microstructured chips can be prepared using standard microfabrication techniques known in the art (e.g., photolithographic processes, etc.). Once formed, the channel surfaces can be oxidized. The oxidization of the device surface can provide a site which acts as the foundation for hydroxylation which is accomplished by boiling in ethanol. The hydroxylation can provide a binding site for silanization which can be performed for 16 hours in an ethanol silane solution. The device can then functionalized with recognition elements (e.g., antibodies), which bind to the silanes, by incubating at physiological temperatures and then rinsed to remove any unbound antibodies.

After surface functionalization with antibodies the chips can be stored in sterile phosphate buffered saline (st-PBS) or used immediately. Devices stored are cataloged and prepared for testing at a later date in increments of 12 hours up to 72 hours, then 24 hours up to 10 days. Finally, one week increments will be studied for long term comparison between units.

Characterization can be performed by wiring a device in to the spring pin connection rig which is connected to a Keithley 5482 source meter unit (SMU). The pins make connection to the source and drain of the three HFETs on each die. A potential bias can swept from 0-1 V and the current is measured as a function of voltage. The signal is collected via labview for continued analysis. After this, the target antigen (e.g., Salmonella Ent.) can introduced and set aside for 5-15 minutes. The die can then be rinsed, and the signal can be collected again.

### Example 2: Process Control Sensor for the Detection and Quantification of Listeria

Listeria bacteria is a significant public health concern. Many species of Listeria have been identified, of which several can cause disease in humans. The most troublesome species is Listeria monocytogenes which is responsible for numerous infections/year in the U.S. alone. While the infection rate from Listeria is lower than for Salmonella, the mortality rate for Listerosis (i.e., an infection from Listeria) approaches 50%, and the per-case medical cost is over $1M. Listeria is endemic in the environment thrives in the cool wet conditions frequently found in food processing plants. Listeria is being found with increasing frequency in a wide range of raw and processed foods. The accurate detection of Listeria during harvest, processing, manufacturing and shipping are critical to preventing its spread. A common source of Listeria contamination is the wash systems commonly used in food processing facilities. Food processors that handle fresh produce will often have one or more wash stations. The wash water is recycled for economic reasons and, if contaminated, can spread Listeria to the food itself. Since existing diagnostics (PCR, ELISA) are time consuming (>48 hrs to result) and require long incubation times (>12 hrs) they are not suitable for real-time bacterial contamination monitoring in process water.

In a process control application, the sensor described herein can be modified to operate remotely from the reader through the use of an intermediary adapter circuit incorporating the signal conditioning circuits, power and wireless communication capabilities of the reader (FIG. 4) for installation at the point of measurement. The sensor cartridge can be modified to allow for continuous contact with the target (e.g., wash water). The reader itself can be adapted to connect with several process control sensor adapters through a wireless interface, as is known in the art.

The devices, systems, and methods of the appended claims are not limited in scope by the specific devices, systems, and methods described herein, which are intended as illustrations of a few aspects of the claims. Any devices, systems, and methods that are functionally equivalent are intended to fall within the scope of the claims. Various modifications of the devices, systems, and methods in addition to those shown and described herein are intended to fall within the scope of the appended claims. Further, while only certain representative devices, systems, and method steps disclosed herein are specifically described, other combinations of the devices, systems, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A system for detecting an analyte of interest comprising:
(a) a sensor cartridge comprising a chip, wherein the chip comprises an active sensor, a reference sensor; and
(b) a cartridge reader comprising:
(i) a receiving unit configured to physically receive the sensor cartridge, the receiving unit further comprising receiving components that operably connect to the active sensor and the reference sensor; and
(ii) a microprocessor configured to analyze an electrical property of the active sensor and an electrical property of the reference sensor to detect the analyte of interest;
wherein the active sensor comprises:
a substrate;
a channel disposed on the substrate, wherein the channel is substantially impermeable to ions under physiological conditions;
a source electrode and a drain electrode electrically connected to the channel, wherein the source electrode and the drain electrode are formed to be separate such that the channel forms a path for current flow between the source electrode and the drain electrode; and
a recognition element for the analyte of interest immobilized on the surface of the channel;
wherein the distance between the recognition element and the channel is configured such that association of the analyte of interest with the recognition element induces a change in the electrical properties of the channel; and
wherein the reference sensor comprises:
a substrate;
a channel disposed on the substrate, wherein the channel is substantially impermeable to ions under physiological conditions;
a source electrode and a drain electrode electrically connected to the channel, wherein the source electrode and the drain electrode are formed to be separate such that the channel forms a path for current flow between the source electrode and the drain electrode; and
a passivating layer disposed on the surface of the channel.

2. The system of claim 1, wherein the cartridge reader further comprises a display configured to display an output from the microprocessor related to the detection of the analyte of interest.

3. The system of any of claims 1-2, wherein the cartridge reader further comprises a communication interface configured to transmit data from the microprocessor to a remote computing device.

4. The system of claim 3, wherein the communication interface comprises a wireless communication interface.

5. The system of any of claims 1-4, wherein the cartridge reader further comprises an input device configured to provide user inputs to the microprocessor.

6. The system of claim 5, wherein the input device comprises a barcode scanner.

7. The system of claim 5, wherein the input device comprises a keypad.

8. The system of any of claims 5-7, wherein the microprocessor is configured to correlate user inputs from the input device to an electrical property of the active sensor, an electrical property of the reference sensor, an output from the microprocessor related to the detection of the analyte of interest, or a combination thereof.

9. The system of any of claims 1-8, wherein the channel comprises a Group III-nitride heterojunction,
wherein the Group III-nitride heterojunction comprises a first Group III-nitride layer and a second Group III-nitride layer, and
wherein the first Group III-nitride layer and the second Group III-nitride layer have different bandgaps, such that a two-dimensional electron gas is generated inside the Group III-nitride heterojunction.

10. The system of claim 9, wherein the first Group III-nitride layer comprises a material selected from the group consisting of GaN, InN, InGaN, AlGaN, and combinations thereof.

11. The system of claim 9 or 10, wherein the second Group III-nitride layer comprises a material selected from the group consisting of AlGaN, AlN, InAlN, GaN, and combinations thereof.

12. The system of any of claims 9-11, wherein the first Group III-nitride layer comprises GaN and the second Group III-nitride body comprises AlGaN.

13. The system of any of claims 1-12, wherein the recognition element is immobilized on the surface of the channel via a linking group.

14. The system of claim 13, wherein the linking group is selected such that the distance between the recognition element and the surface of the channel is less than about 10 nm.

15. The system of any of claims 13-14, wherein the linking group comprises a polyvalent linking group.

16. The system of claim 15, wherein the linking group is derived from a polyvalent linker selected from the group consisting of (3-aminopropyl)triethoxysilane (APTES), (3-glycidyloxypropyl)trimethoxysilane, (3-mercaptopropyl) trimethoxysilane, vinyltrimethoxysilane, allyltrimethoxysilane, (3-bromopropyl) trimethoxysilane, triethoxyvinylsilane, triethoxysilane aldehyde, and combinations thereof.

17. The system of any of claims 13-14, wherein the linking group comprises a monovalent linking group.

18. The system of claim 17, wherein the monovalent linking group comprises an alkyl group having from 1 to 6 carbon atoms in its backbone.

19. The system of claim 17 or 18, wherein the monovalent linking group is derived from a linker which comprises a monoalkoxysilane moiety.

20. The system of claim 17 or 18, wherein the monovalent linking group is derived from a linker which comprises a monohalosilane moiety.

21. The system of any of claims 17-20, wherein the monovalent linking group is derived from a monovalent linker selected from the group consisting of (3-aminopropyl) dimethylethoxysilane (APDMES), (3-glycidoxypropyl)dimethylethoxysilane, (4-chlorobutyl)dimethylchlorosilane, and combinations thereof.

22. The system of any of claims 1-21, wherein the recognition element is selected from the group consisting of antibodies, antibody fragments, peptides, oligonucleotides, DNA, RNA, aptamers, and organic molecules.

23. The system of any of claims 1-22, wherein the recognition element selectively associates with the analyte of interest.

24. The system of any of claims 1-23, wherein the recognition element comprises an immunoglobulin G (IgG) antibody.

25. The system of any of claims 1-24, wherein the recognition element comprises a single-chain variable fragment (scFv).

26. The system of any of claims 1-25, wherein the recognition element comprises a single-domain antibody (sdAb).

27. The system of any of claims 1-26, wherein the recognition element comprises an antibody that selectively associates with Salmonella enterica, E. coli, or Listeria.

28. A method of detecting an analyte comprising applying a sample comprising the analyte to the chip of the sensor cartridge of the system defined by any of claims 1-27, and determine a change in an electrical property of the active sensor, wherein the change in the electrical property indicates the presence of the analyte.

29. The method of claim 28, wherein the analyte comprises a food-borne pathogen.

30. The method of claim 28 or 29, wherein the analyte comprises Salmonella enterica, E. coli, or Listeria.
